(19) United States
(12) Patent Application Publication
(10) Pub. No.: US 2018/0135137 A1
(43) Pub. Date: May 17, 2018

(54) NUCLEIC ACID PROBES

(71) Applicant: CYTOTEST INC., Rockville, MD (US)

(72) Inventors: Reinhard Ebner, Gaithersburg, MD (US); Hua ZOU, Rockville, MD (US); Chi-Chung Cheng, Rockville, MD (US)

(73) Assignee: Cytotest, Inc., Rockville, MD (US)

(21) Appl. No.: 15/862,343

(22) Filed: Jan. 4, 2018

Related U.S. Application Data
(63) Continuation of application No. PCT/US16/40920, filed on Jul. 4, 2016.
(60) Provisional application No. 62/188,701, filed on Jul. 5, 2015.

(51) Int. Cl. C12Q 1/6886

(52) U.S. Cl. CPC C12Q 1/6886

(57) ABSTRACT

The present embodiments relate to nucleic acid probes for detecting PTEN, ZBTB20-LSAMP, and LSAMP mutations. The nucleic acid probes are particularly useful for detecting deletions in the PTEN and LSAMP genes as diagnostics for prostate cancer, especially aggressive forms of prostate cancer for which treatment is indicated. The probes are particularly useful for in situ hybridization to chromosomes present in tissue samples.

### CROSS-REFERENCE TO RELATED APPLICATIONS

This application is a continuation of International Application No. PCT/US2016/25870, filed Apr. 4, 2016, which claims the benefit of U.S. Provisional Application No. 62/188,701, filed Jul. 5, 2015, entitled “NUCLEIC ACID PROBES,” which are hereby incorporated by reference in their entirety.

### BRIEF DESCRIPTION OF THE SEVERAL VIEWS OF THE DRAWINGS

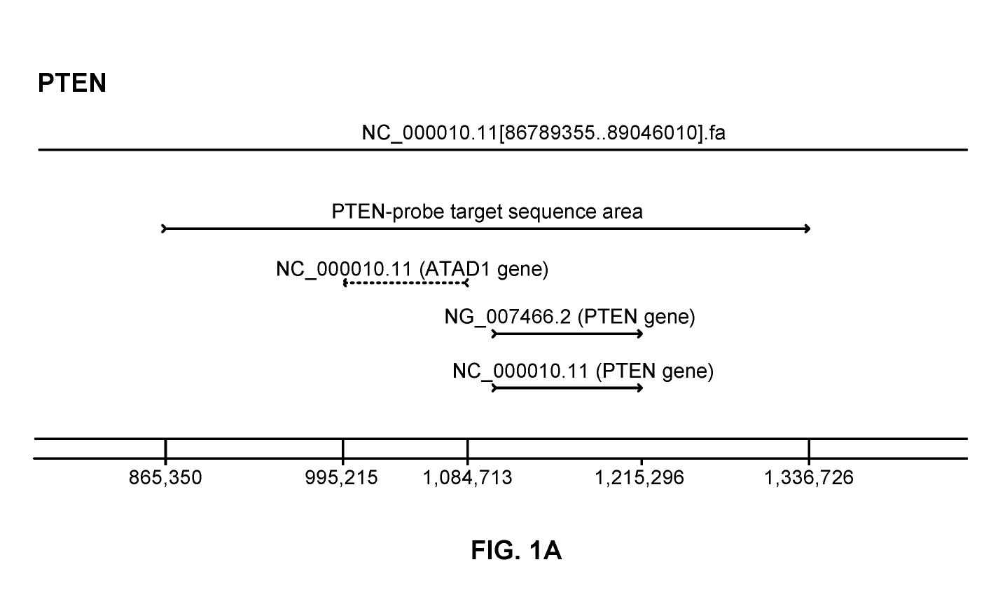

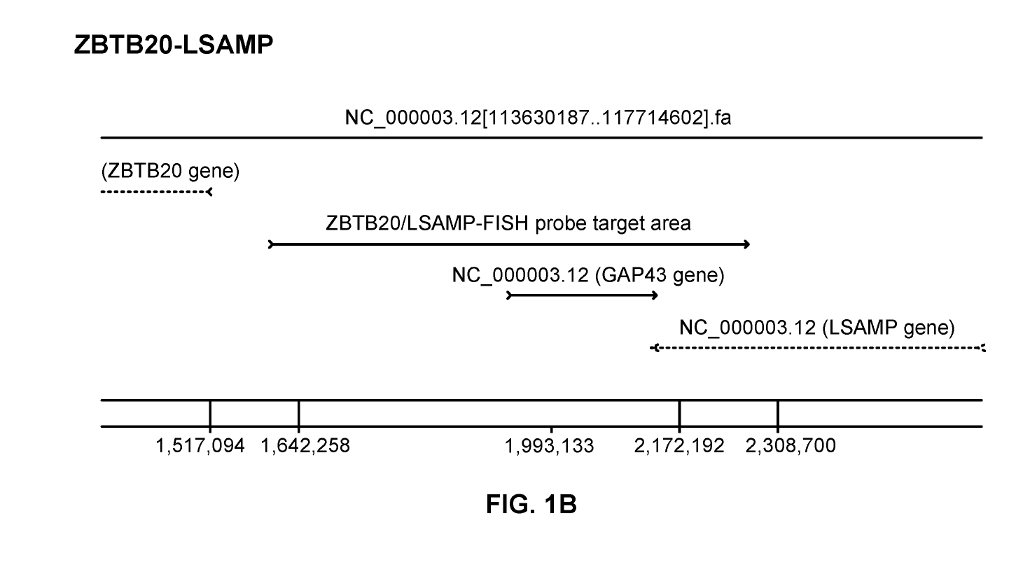

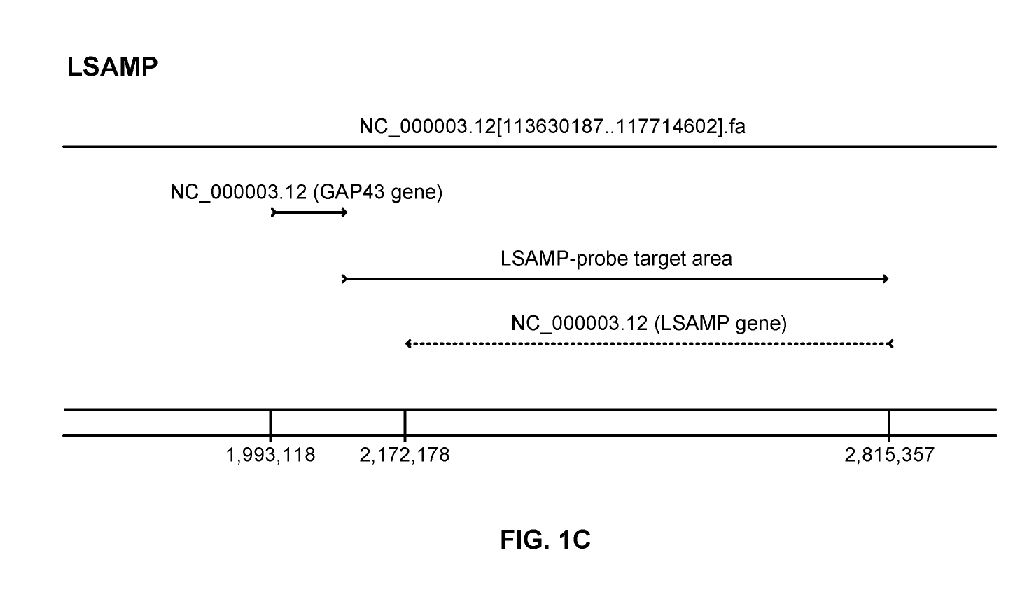

Example FIG. 1A is a diagram showing approximate location of a PTEN-FISH probe target area as per an aspect of an embodiment of the present invention.

Example FIG. 1B is a diagram showing approximate location of a first-generation LSAMP-FISH probe target area as per an aspect of an embodiment of the present invention.

Example FIG. 1C is a diagram showing approximate location of a second-generation LSAMP-FISH probe target area as per an aspect of an embodiment of the present invention.

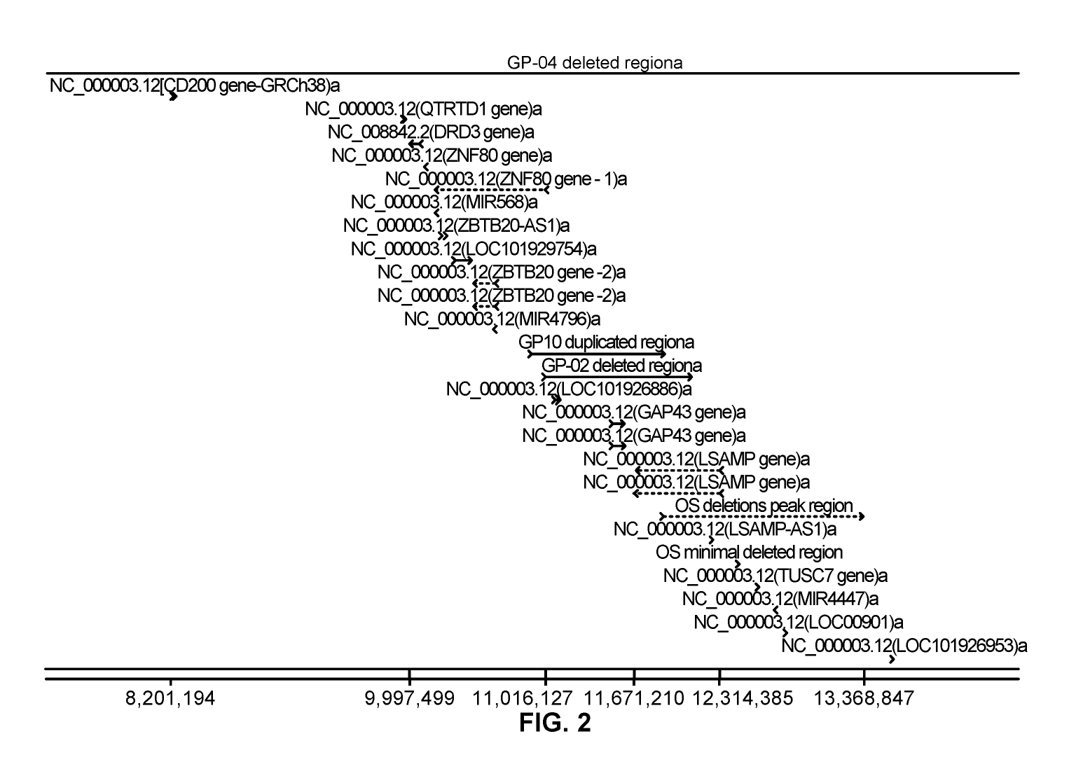

Example FIG. 2 is a schematic illustration of LSAMP deletion locations and sizes as per an aspect of an embodiment of the present invention.

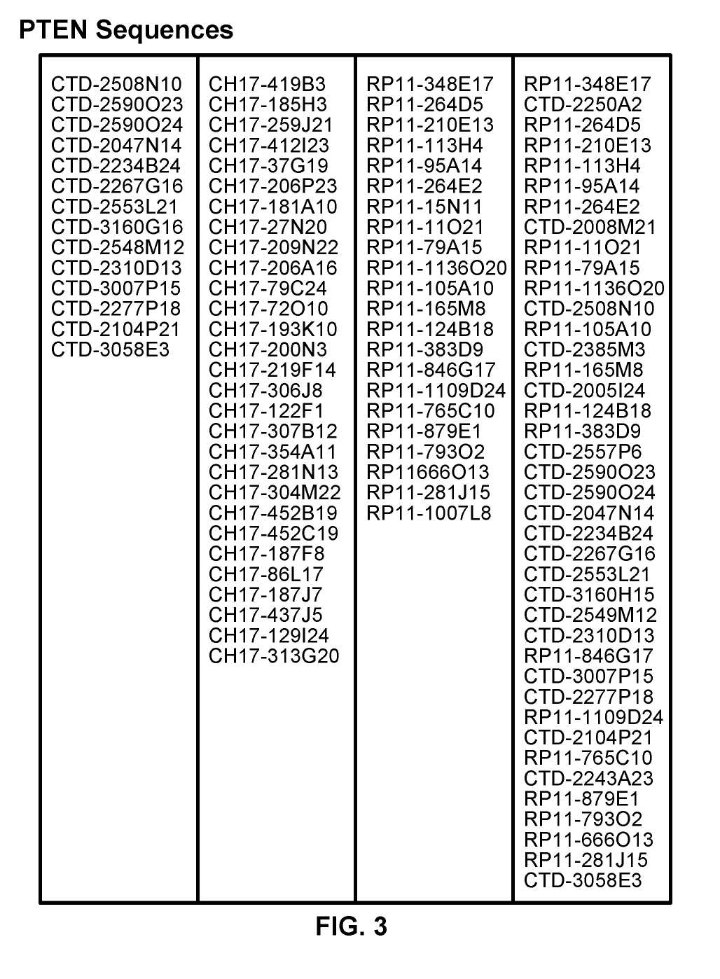

Example FIG. 3 is a table of example PTEN sequences for preparing nucleic acid probes as per an aspect of an embodiment of the present invention.

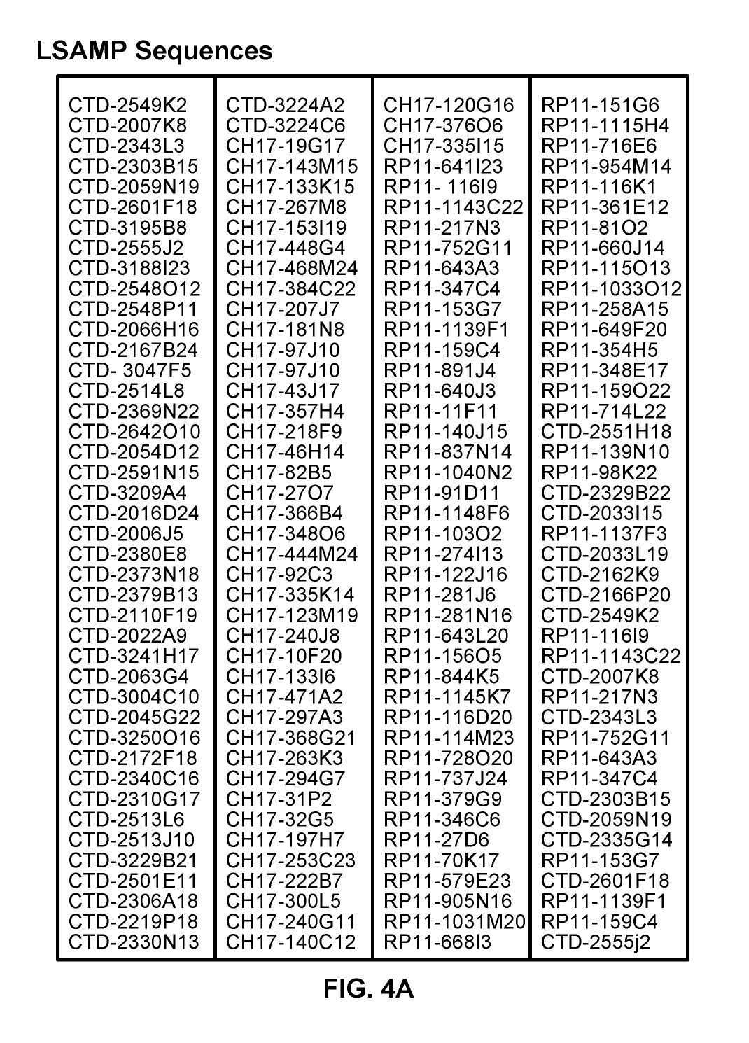

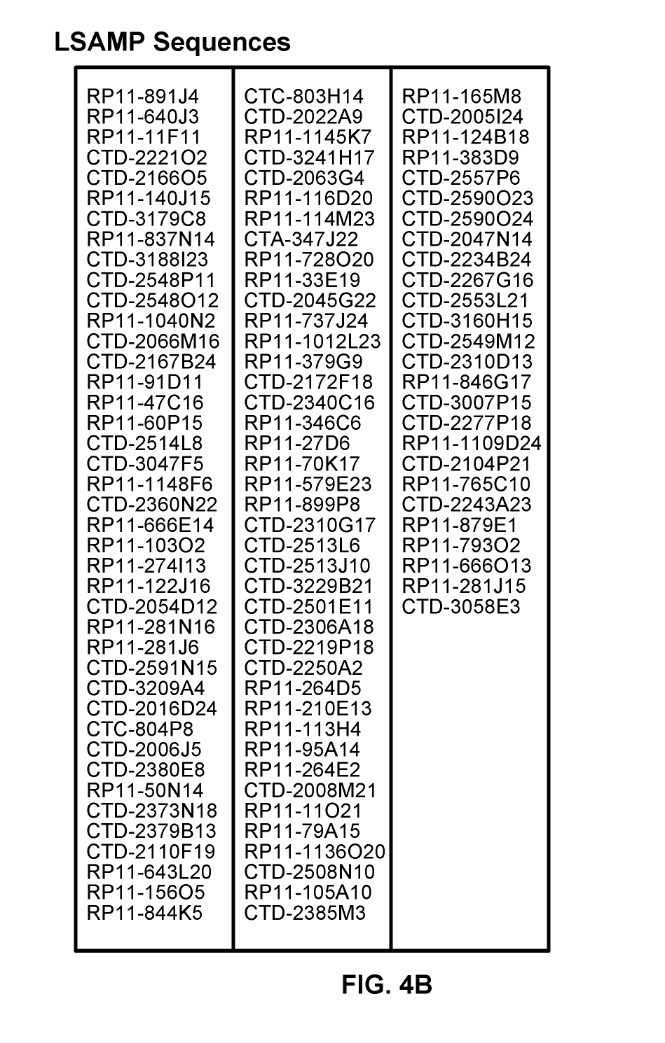

Example FIG. 4A is a table of example LSAMP sequences for preparing nucleic acid probes as per an aspect of an embodiment of the present invention.

Example FIG. 4B is a table of example LSAMP sequences for preparing nucleic acid probes as per an aspect of an embodiment of the present invention.

### DETAILED DESCRIPTION OF EMBODIMENTS

The present invention relates to nucleic acid probes for detecting nucleic acid sequences. The sequences may be present in various samples, including in tissues, cells, organelles, chromosomes, biopsy samples, tissues present on slides, skin, hair, environmental, soil, clothing, forensic samples, etc. The probes may be especially useful in diagnosing prostate cancer, particularly an aggressive form of prostate cancer. The probes may be useful for detecting deletions in the PTEN and LSAMP genes as diagnostic for prostate cancer. Particularly when LSAMP is deleted in a patient, the patient may be an immediate candidate for one of the conventional therapeutic interventions utilized in prostate cancer since LSAMP associated prostate cancer may be aggressive and indicate a need for therapeutic intervention.

Generally, a target acid nucleic acid is selected for detection. For example, it may be desired to determine whether a gene has been amplified, rearranged or deleted in a chromosome, or translocated to another chromosome. In the case of amplification, the copy number of a gene may be increased in comparison to other genes present on the chromosome. Amplification generally involves multiplication of a region of the chromosome resulting in an increase in the copy number of the genes in the amplified region. Genes can also become rearranged, including by deletions, insertions, fusions, translocations, and other aberrations, e.g., involving other genes and chromosomal regions.

The probes of the present embodiments may be useful for detecting any of the above-mentioned genomic changes in a genome. In addition, the probes may be useful in detecting aneuploidy, such as trisomy, where karyotyping is typically utilized to detect genetic abnormalities.

FIG. 1A is a diagram showing approximate location of a PTEN-FISH probe target area, relative to the position of the PTEN gene. The probe may cover the entire gene region. An adjacent gene is shown for orientation. FIG. 1B shows the approximate location of a first-generation LSAMP-FISH probe target area: the probe covers a genomic region reaching from between (but excluding) the ZBTB20 and GAP43 genes to beyond the 3′ third of the LSAMP gene. FIG. 1C shows the approximate location of a second-generation LSAMP-FISH probe target area: the probe is centered on the LSAMP gene and covers most of the GAP43-LSAMP intergenic region as well as most of (more than 95%) the LSAMP gene itself.

FIG. 2 is a schematic illustration of LSAMP deletion locations and sizes. Three example representative regions affected in African American prostate cancer cases are shown: GP-02 (ca. 1.5 Mb deletion), GP-04—large deletion, ca. 22 Mb; only the center of this deletion is seen in the figure), GP-10 (ca. 2 Mb inversion/duplication). Also indicated are the peak and minimal regions of the LSAMP region reported in osteosarcoma according to Mol Cancer. 2014; 13: 93 (doi: 10.1186/1476-4598-13-93). Several genes flanking LSAMP are shown for orientation. LSAMP is the only known gene in this genomic region that overlaps with all of the deletions shown in the figure.

FIG. 3 (PTEN) and FIG. 4 (LSAMP) show example BAC clones for preparing nucleic acid probes in accordance with the present embodiments that may be used for detecting deletions of the PTEN and LSAMP genes, respectively.

To make a nucleic acid probe in accordance with the present invention, a specific region is selected as a target to be detected. The target can be of any desired size, e.g., the entire region, a part of a region, or a specific gene or genes. Once the target is identified, the nucleic acid from the region is obtained for preparation of the nucleic acid probe. For example, when a deletion of the LSAMP gene is to be detected, nucleic acid molecules (e.g., BAC clones) may be selected which span a part or the entire region of the gene as desired, e.g., covering 50%, 60%, 70%, 80%, 90%, 95%, etc., of the gene, and values in between.

There are a variety of difference sources from which the nucleic acid can be obtained. These include, but are not limited to, BAC (bacterial artificial chromosome) libraries, YAC (yeast artificial chromosome) libraries, PCR (polymerase chain reaction) product fragments, bacteriophage libraries, plasmid libraries, cDNA libraries, genomic libraries, libraries made from dissected chromosomal regions.

The probe may be prepared by any suitable method. Generally, once the nucleic acid to be used as a probe source is obtained, it will be amplified to increase its amount, e.g., by PCR, nick-translation, random priming, etc.

Probes prepared in accordance with the above-mentioned methods may incorporate naturally-occurring and non-naturally occurring nucleotides. Examples of non-naturally occurring nucleotides useful in the present include, but are not limited to, nucleotides which are disclosed in U.S. Pat. Nos. 5,476,928, 5,449,767, and 5,328,824.

Probes may be labeled with detectable labels to enable detection of the probe. The probe can be labeled prior to its hybridization with a target, during hybridization, or after hybridization. Detectable labels and methods of labeling nucleic probes are well known in the art.

Useful detectable labels include, but are not limited to: fluorescent dyes, biotin, enzymes, fluorescein, Texas Red, DNP, fucose. Labeling methods are well known in the art.

The nucleic acid probes of the present invention are non-naturally occurring. Specifically, in preferred embodiments, the probes are not directed to contiguous and connected chromosomal regions, but rather are fragmented portions of the desired region. For example, for region 10q23 to detect PTEN deletions, the probe does not comprise molecules which are continuous or contiguous with a genomic sequence from that region, but rather contains non-continuous fragments from it.

In addition to not being a continuous region, the probe preferably does not contain equal representations or proportions of each sub-region within the target region. For example, if chromosome band 10q23 comprising the PTEN gene is selected, the probe will contain fragments of it in unequal quantities, i.e., if the region has ten different fragments within it, fragment 1 may be present in 1× quantity, fragment 2 in 2× quantity, fragment 3 in 3× quantity, fragment 4 in 4× quantity, and so on. Such unequal representations from the molecules as they occur in nature result from the selection of non-overlapping molecules from which to prepare the probe, and subsequent amplification reactions which unequally amplify parts of the target nucleic acid. The same applies to detecting deletions of the LSAMP gene.

Hybridization

Once a probe is produced as described above, it may be used to detect the target nucleic acid in a sample. Generally, the probe may be used as a hybridization probe in any suitable format. Formats include, without limitation, liquid hybridization, PCR, Southern, Northern, microarrays, microscope slides, paraffin sections, cryosections.

Hybridization conditions are well known in the art. See, e.g., Wangsa et al., Am. J. Pathol., 175(6): 2637-2645, December 2009.

As indicated above, the probe may be pre-labeled, such that after hybridization is complete and unbound probe is washed away, the probe can be immediately detected. In another embodiment, detectable label can be added to the probe after its bound to the target nucleic acid.

In Situ Hybridization

In situ hybridization (ISH) is a technique that involves hybridizing a probe to a target nucleic acid in which the target is present in a tissue section (paraffin, plastic, cryo, etc.), cells, embryos, etc. In this technique, the target is detected in situ in the location where it is normally found. For example, the target can be detected in the cell cytoplasm, in an organelle (e.g., mitochondria), or in the chromosomal DNA. The chromosomal DNA in general is an intact chromosome that can be present in the tissue section or cell in its intact form or it can be isolated. In each case, the sample containing the target may be treated in such a way that the probe can access the target chromosome or chromosome fragment, hybridize to it, and then be detected. When the probe is fluorescently labeled, the technique is known as fluorescence in situ hybridization (FISH).

ISH can be performed with one or more detectable labels. For example, M-FISH (multi-fluor or multi-color or multispectral FISH) is a technique in which multiple probes, each of which binds to a different DNA sequence and each of which bears a different detectable label, is used to detect multiple different sequences on the same sample, for example, on the same chromosome. M-FISH may be useful for looking at chromosome rearrangements or translocations, or looking at independent loci in the same sample. See, e.g., U.S. Pat. No. 5,880,473 for the use of multiple filters in M-FISH. For SKY (spectral karyotyping), in which each chromosome pair is visualized in a different color, see, e.g., Schröck E, du Manoir S, Veldman T, Schoell B, Wienberg J, Ferguson-Smith M A, Ning Y, Ledbetter D H, Bar-Am I, Soenksen D, Garini Y, Ried T. Multicolor spectral karyotyping of human chromosomes. Science 273:494-497, 1996.

A given dye is characterized by an excitation (absorption) spectrum and an emission spectrum. The excitation and emission spectra are also sometimes referred to as the excitation and emission bands. When the dye is irradiated with light at a wavelength within the excitation band, the dye fluoresces, emitting light at wavelengths in the emission band. Thus, when the sample is irradiated with excitation radiation in a frequency band that excites a given dye, portions of the sample to which the probe labeled with the given dye is attached fluoresce. If the light emanating from the sample is filtered to reject light outside the given dye's emission band, and then imaged, the image nominally shows only those portions of the sample that bind the probe labeled with the given dye.

The term “hybridization” refers to the specific binding of a nucleic acid to a complementary nucleic acid via Watson-Crick base pairing. The term “in situ hybridization” refers to specific binding of a nucleic acid to a target nucleic acid in its normal place in a sample, such as on metaphase or interphase chromosomes. The terms “hybridizing” and “binding” are used interchangeably to mean specific binding between a nucleic acid probe and its complementary sequence.

The term “chromosomal region” means a contiguous length of nucleotides in the genome of an organism. A chromosomal region may be in the range of 10 kb in length to less than a complete chromosome of an entire chromosome, e. g., 100 kb to 10 MB for example. FISH probes are most typically in the 50 kpb to 1000 kbp length range.

The term “in situ hybridization conditions” refers to conditions that facilitate hybridization of a nucleic acid to a complementary nucleic acid in an intact chromosome. Suitable in situ hybridization conditions may include both hybridization conditions and optional wash conditions, which include temperature, concentration of denaturing reagents, salts, incubation time, etc. Such conditions are known in the art.

FISH probes can be prepared according to standard procedures. See, e.g., Bolland, D. J., King, M. R., Reik, W., Corcoran, A. E., Krueger, C. Robust 3D DNA FISH Using Directly Labeled Probes. J. Vis. Exp. (78), e50587, doi:10.3791/50587 (2013).

Probe Selection

Determination of the specific probe to be used to detect the target sequence can be accomplished routinely. Probe property may be selected based on one or more of the following factors, duplex melting temperature, hairpin stability, GC content, probe complementary to an exon, probe complementary to a gene, probe complementary to intron, probe complementary to multiple regions in the genome and a proximity score. In certain embodiments, the probes may be comprised of fragments which were selected for different properties, such as the factors mentioned above. For example, fragments can be selected based on different factors, such as GC content or hairpin stability, and then pooled to make the final nucleic acid probe. See US 2011/003935 A1 for methods of selecting probes.

When a certain chromosomal region is targeted, a set of tiled or overlapping candidate nucleic acids may be selected, such as tiled YAC or BAC clones. Such tiled or overlapping nucleic acids may be constructed to unique sequences in the desired chromosomal regions. Because of the tiling or overlapping, the regions of overlap are in greater quantity than other non-overlapping regions, and thus are represented in higher amounts than in the native chromosome, particularly when amplified using a polymerase or other amplification method.

When ISH probes are made from artificial chromosomes, such as yeast artificial chromosomes (YAC), bacterial artificial chromosomes (BAC) and phage artificial chromosomes (PAC), etc., nucleotide repeats and repetitive sequences are usually present which can produce non-specific fluorescent signal and reduce the ISH detection specificity and sensitivity. Methods to reduce hybridization are known in the art, and include adding repetitive sequences to the hybridization mixture or making ISH probes that lack such sequences.

Probes may be tested to avoid using probes hybridizing to repetitive and repeat sequences. Probes can be produced using sets of various oligonucleotides which avoid repetitive sequences present in a flanking region. Such sets can be distinctly labeled, with separate or distinct reporter molecules for each probe (or set of oligonucleotides) that is aimed at the respective flanking region. Such probes can each consist of multiple labeled oligonucleotides, each hybridizing to a distinct area in a region which lacks repetitive sequences. One probe can, for example, contain from 10 up to 200 of such oligonucleotides, preferably from 50-150, each oligonucleotide, for example, being 10-20 nucleotides long.

As mentioned, the probes of the present invention can be produced by any suitable or known method. For example, probes can be produced using set of oligonucleotides that amplify unique, non-repetitive regions. See, e.g., WO 2014036525 A1.

Probes designed for translocations, break points, inversions, and other chromosomal rearrangements can be produced routinely. Generally, chromosomal regions flanking a breakpoint are selected. Each flanking region is labeled differently.

Probes can also be provided to identify translocations. In such cases, a balanced pair of nucleic acid probes can be produced. The probes in said pair are comparable or balanced in that they are designed to be of for example comparable size or genomic length with the final aim of facilitating the generation of signals of comparable intensity. In addition, said probes can be comparably labelled with reporter molecules resulting in signals of comparable intensity. In addition, the probes may each be labelled with a different fluorochrome, facilitating detection on one spot of different color when they co-localize when no aberration is detected. In addition, probes can be selected to react with a chromosome, at respective complementary sites that are located at comparable distances at each side of a breakpoint or breakpoint cluster region of a chromosome. The distinct and balanced pair of nucleic acid probes provided by the invention entails probes that are for example of comparable size or genomic length, each probe of the pair for example being from 1 to 10 kb, or 7 to 15 kb, or 10 to 20 kb, or 15 to 30 kb, or 20 to 40 kb, or 30 to 50 kb, or 40 to 60 kb, or 50 to 70 kb, or 60 to 80 kb, or 70 to 90 kb, or 80 to 100 kb, or 100 to 500 kb or more in length. By using such a distinct and balanced pair of probes flanking a breakpoint region and not overlapping the corresponding fusion region, false-positive diagnosis in hybridization studies is avoided.

Labeling

The labeling may be done in any one of a number of convenient ways. For example, in certain cases, the probes may be labeled by chemically conjugating one or more labels to the one or more double stranded polynucleotides, e.g., using the Universal Linkage System (ULS™, KREATECH Diagnostics; van Gijlswijk et al Universal Linkage System: versatile nucleic acid labeling technique Expert Rev. Mol. Diagn. 2001 1:81-91). Alternatively, the labeling may be done using nick translation, by random priming, or any other suitable method described in Ausubel et al. (Short Protocols in Molecular Biology, 3rd ed., Wiley & Sons, 1995), or Sambrook et al. (Molecular Cloning: A Laboratory Manual, Third Edition (2001) Cold Spring Harbor, N.Y.). In certain cases, the one or more double stranded polynucleotides are labeled at multiple sites and not labeled by end labeling. As would be apparent embodiments of the method that use other labeling methods (e.g., nick translation or random priming) will produce products that differ in sequence and representation of sequence.

PTEN Probes

Phosphatase and tensin homolog (PTEN) is a tumor suppressor gene that is mutated in a large number of cancers at a high frequency. The PTEN gene is located on the long (q) arm of chromosome 10 at position 23.3.

PTEN is assigned unique identifier codes by HGNC and Entrez Gene which are HGNC:9588 and Entrez Gene:5728, respectively. The accession number of representative PTEN nucleic acid and polypeptide sequences is NM 000314.4, GT:257467557, which sequences are incorporated by reference in their entirety. The chromosomal location of the PTEN gene is 10q23. PTEN is located at 87,863,438-87,971,930 bp in GRCh38.p2 coordinates which is represented by ENSG00000171862. These sequences are incorporated by reference in their entirety.

To produce probes against the PTEN genes, overlapping and non-overlapping chromosomal segments may be selected and routinely amplified to produce a non-naturally occurring probe composition. As indicted above, nucleic acid to produce such probes can be obtained from any suitable source, such as a BAC clones, YAC clones libraries, etc. One or more, such as e.g. 2, 3, 5, 7, 10 clones can be pooled together to create a probe. The clones can be amplified separately or pooled and then amplified. When non-overlapping clones are utilized, the clones can be selected such that the entire gene is represented, or only a part of it, where the clones collectively lack parts of the gene due to the selection of non-overlapping segments.

FIG. 3 shows a list of publicly available BAC clones that include sequences from PTEN, or regions adjacent to it. The sequences and clones are incorporated by reference. Two or more clones can be selected to make a probe, e.g., where the clones are amplified separately or in combination by nick-translation, random primer, etc. All combinations of clones to make PTEN probes are covered by the present invention. The clones are chosen such that, when detectably labeled, the absence of hybridization to an in situ prostate sample (i.e., to the chromosome) indicates that the PTEN gene has been deleted, and thus is diagnostic of the prostate cancer.

The probes can be useful to detect amplification of the PTEN gene or deletion of the gene. For example, gene deletion occurs in certain prostate cancers and therefore proves suitable to detect gene deletions, and such are useful for diagnostic purposes. Accordingly, the invention comprises a method for detecting the presence or absence of PTEN in a biological sample comprising nucleic acid. ISH probes are particularly useful for this purpose.

ZBTB20 and LSAMP Probes

Another useful probe is based on a genomic re-arrangement that occurs in chromosomal region 3q13 which involves the ZBTB20 (zinc finger and BTB containing 20) and LSAMP (limbic system associated membrane protein) genes. The ZBTB20/LSAMP genomic re-arrangement can be a gene fusion between the ZBTB20 gene and the LSAMP gene, a gene inversion, a gene deletion, or a gene duplication. The rearrangement is useful to detect prostate cancer or an increased likelihood to develop prostate cancer or characterizes the prostate cancer in the subject as being an aggressive form of prostate cancer or as having an increased risk of developing into an aggressive form of prostate cancer. In one aspect, a gene fusion of ZBTB20 and LSAMP is detected in a biological sample from a subject. A probe to detect such a fusion can comprise sequences from both genes. The probes can be used in combination, or each alone, to detect and diagnose cancer, such as prostate cancer.

The unique identifier code assigned by HGNC for the ZBTB20 gene is HGNC:13503. The Entrez Gene code for ZBTB20 is 26137. There are at least 7 alternative transcript variants detected for ZBTB20 and at least four distinct promoters that can initiate transcription from at least four distinct sites within the ZBTB20 locus, producing four variants of exon 1 of ZBTB20: El, EIA, EIB, and EIC. Representative nucleotide and amino acid sequences of ZBTB20 variant 1 are known and represented by the NCBT Reference Sequence NM_001164342.1 and G1:257900532, which sequences are incorporated by reference in their entirety. Variant 2 differs from variant 1 in the 5′ untranslated region, lacks a portion of the 5′ coding region, and initiates translation at a downstream start codon, compared to variant 1. The encoded isoform (2) has a shorter N-terminus compared to isoform 1. Variants 2-7 encode the same isoform (2). Representative nucleotide and amino acid sequences of ZBTB20 variant 2 are known and represented by the NCBT Reference Sequence NM_0I5642.4, GI:257900536, which sequences are incorporated by reference in their entirety. The chromosomal location of the ZBTB20 gene is 3q13.2. The genomic sequence included NC_000003.12 (114314500 to 115147280, complement), which is incorporated by reference in its entirety.

The unique identifier code assigned by HGNC for the LSAMP gene is HGNC:6705. The Entrez Gene code for LSAMP is 4045. The nucleotide and amino acid sequences of LSAMP are known and represented by the NCB1 Reference Sequence NM_002338.3, GJ:257467557, which sequences are incorporated by reference in their entirety. The chromosomal location of the LSAMP gene is 3q13.2-q21. The genomic sequence for LSAMP starts from 115,521,210 bp from pter and ends at 117,716,095 bp from pter (reverse strand), which sequences are incorporated in their entirety. Other reference sequences include RefSeq DNA sequence at NCBI GenBank: NC_000003.11, NT_005612.17, and NC_018914.2, which sequences are incorporated by reference in their entirety. Gene information and sequence is located at ENSG00000185565. The genomic region can also include 115,802,363-117,139,389 based on Ensembl release 80 (May 2015).

To detect a gene fusion between ZBTB20 and LSAMP, sequences from each gene can be used. The sequences from each gene can be labeled with a different label such when a fusion is present in a nucleic acid sample, the labels appear to be adjacent to each other, and when absent, the labels are separated from each other and appear as distinct detectable spots on a chromosome utilized in an ISH method.

LSAMP Rearrangements and Deletions

The LSAMP gene, or portions of it, can be deleted in patients with aggressive prostate cancer, i.e., where the patient requires treatment. The patient can have been diagnosed with prostate cancer, and of African, Asian, European, or South American descent, preferably of African descent. The deletion can extend from the GAP43 gene and into the LSAMP gene (sequences present in NC_000003.12), including the DNA between the two genes. The deletion can include a part of LSAMP, e.g., the entire genomic sequence, or regions of it, such as coding or non-coding regions. A useful probe can include DNA from the entire LAMP gene and the DNA between LSAMP and the GAP43 genes, optionally including a portion of GAP43 sequence as well, e.g., from 115,400,000 (including GAP43 sequence), from 115,405,000 (end of GAP43 sequence), 115,410,000 to 115,521,210 (start of LSAMP gene), from 115,405,000 to 117,716,095, from 115,802,363-117,139,389 (Ensembl release 80, May 2015), etc.

A useful probe can be made by selecting DNA, such as from a BAC clone, where two or more of the DNAs overlap with each other in such a way that the completed probe contains a higher representation of the overlapped region than regions which show no overlap. For instance, a probe can be designed utilizing overlapping middle regions of the LSAMP gene and non-overlapping 3′ and 5′ regions.

FIG. 4A and FIG. 4B show a list of publicly available BAC clones that include sequences from LSAMP or directly adjacent to it. Two or more clones can be selected to make a probe, e.g., where the clones are amplified separately or in combination by nick-translation, random primer, etc. FIG. 2 shows the location of selected sequences in deleted regions from patients with LSAMP-associate prostate cancer (GP-04, GP-10, GP-02). The common deleted region in all three patients is the LSAMP gene. Overlapping clones from leftmost region can be selected which include LSAMP sequence and/or sequence which overlaps with the GAP43 gene. These can be combined with sequences from middle and rightmost regions of the LSAMP gene, e.g., 3′ end, 5′ end, and middle regions in between. The invention includes all combinations of probes shown in FIG. 2 and listed in FIG. 4. All the sequences are incorporated by reference. The clones are chosen such that, when detectably labeled, the absence of hybridization to an in situ prostate sample (i.e., to the chromosome) indicates that the LSAMP gene has been deleted, and thus is diagnostic of the prostate cancer.

Patients having prostate cancer who have a deletion or rearrangement of the LSAMP or PTEN gene may be treated conventionally with surgery, cryosurgery, radiation, chemotherapy, or hormone therapy. Particularly, patients who have an LSAMP deletion associated with aggressive prostate cancer are candidates for a therapeutic intervention. Interventions include, e.g., radical prostatectomy, radiotherapy, androgen ablation therapy, antiandrogen monotherapy, gonadotropin-releasing hormone (GnRH) agonist, leuprolide, bicalutamide, docetaxel with or without prednisone, mitoxantrone with or without prednisone, cabazitaxel with or without prednisone, abiraterone acetate with or without prednisone, sipuleucel T, enzalutamide, taxane, prednisone, paclitaxel, histone deacetylase inhibitors (HDACi), such as orinostat, romidepsin, and panobinostat. See also Watson et al., Nat Rev Cancer. 2015 December; 15(12): 701-711 (see particularly, Table 1); Wilson et al., Cent European J Urol. 2015; 68(2):165-8. doi: 10.5173/ceju.2015.513. Epub 2015 Apr. 20; Mulders et al., Cancer Immunol Immunother. 2015 June; 64(6):655-63. doi: 10.1007/s00262-015-1707-3. Epub 2015 May 30; Shore, Am J Manag Care. 2014 December; 20(12 Suppl):S260-72; Dreicer, Am J Manag Care. 2014 December; 20(12 Suppl):S282-9; Kolodziej, Am J Manag Care. 2014 December; 20(12 Suppl):S273-81; Rockville (Md.): Agency for Healthcare Research and Quality (US); 2014 December, Report No.: 15-EHC004-EF (incorporated by reference for disclosure relating to therapeutic agents and interventions).

Chromosome Counting Probes

Embodiments of the present invention may also include chromosome counting probes. Such probes can be used to count the chromosomes, e.g., in metaphase spread, and/or to detect specific chromosomes. For example, probes to chromosome centromere regions can be prepared from centromeric DNA using specific primers.

### EXAMPLES

Generate fluorescence labeled DNA probes by Nick Translation. DNA was extracted from identified BAC clones. Labeling was performed in two steps: nick translation introducing aminoallyl-dUTP and chemical coupling of an amine-reactive dye. Specifically, DNase I was used to create single-strand breaks, then DNA polymerase I was used to elongate the 3′ ends of these “nicks”, replacing existing nucleotides with new aminoallyl-dUTP. The fluorescent labeling of the probe was completed by chemical coupling of the dye. Alexa Fluor succinimidyl ester dyes react with the amines of the amino-allyl-dUTP modified DNA, thereby forming fluorescently labeled probes. Standard Ethanol precipitation method was used to isolate the fluorescently labeled probe. The probe pellet was suspended in deionized formamide/dextran sulphate.

DNA FISH Protocol. Cell slides were pretreated in pepsin solution before undergoing fixation in formaldehyde, followed by serial ethanol dehydration. The slides were denatured in formamide/saline sodium citrate (SSC) solution, followed by ice cold dehydrating ethanol series. Probes were denatured at 80° C. followed by a pre-annealing step. Pre-annealed probes were added to the denatured slides. The slides were then cover-slipped and sealed for overnight hybridization in a humidified chamber. After hybridization, slides were washed and dehydrated. At last, the slides were counterstained with anti-fade solution and mounted with coverslip for observation.

FISH Procedure for formalin-fixed paraffin-embedded (FFPE) specimens

Reagents that may be employed to practice one or more embodiments:
Paraffin Pretreatment Reagent Kit (Cat No: CT-ACC112-05):
   Pretreatment Solution (50 ml): store at room temperature (RT)
   Protease Buffer (62.5 ml, pH 2.0): store at RT
   Protease (250 mg): Lyophilized, store at −20° C.
FISH Reagent Kit (Cat No: CT-ACC101-20):
   20× Sodium Chloride-Sodium Citrate Buffer (SSC) Salt: store at RT, avoid humidity
   4′,6-diamidino-2-phenylindole (DAPI) Counterstain: store at 4° C. in the dark
   NP-40 (octylphenoxypolyethoxyethanol, or Nonidet P-40): store at RT
Xylene: store at RT
Ethanol (100%): store at RT
Purified water: store at room RT
Concentrated (12N) HCl: store at room RT

Preparation of Working Solutions

| | | |
|---|---|---|
| Reagents | Amount added | Final Concentration |

1. 20X SSC Solution (pH 7.0)

| | | | |
|---|---|---|---|
| SSC Salt | 66 | g | 20X |
| Deionized H₂O (dH₂O) | 250 | ml | |
| TOTAL | 250 | ml | |

2. Protease Solution

| | | | |
|---|---|---|---|
| Protease, lyophilized | 250 | mg | 4 mg/ml |
| Protease Buffer | 62.5 | ml | |
| TOTAL | 62.5 | ml | |

3. 90% Ethanol

| | | | |
|---|---|---|---|
| Ethanol (100%) | 90 | ml | 90% |
| dH₂O | 10 | ml | |
| TOTAL | 100 | ml | |

4. 70% Ethanol

| | | | |
|---|---|---|---|
| Ethanol (100%) | 70 | ml | 70% |
| dH₂O | 30 | ml | |
| TOTAL | 100 | ml | |

5. Post-hydridization Wash Solution (pH 7.0)

| | | | |
|---|---|---|---|
| 20X SSC Solution | 10 | ml | 2X |
| NP-40 | 300 | μl | 0.3% |
| dH₂O | 90 | ml | |

| | |
|---|---|
| TOTAL | 100 |

FISH Procedure for Paraffin-embedded Tissue Sections

Slide Pretreatment
1. Immerse slides in xylene at RT for 10 minutes. Repeat twice with fresh xylene each time.
2. Dehydrate slides in 100% ethanol at RT for 5 minute. Repeat once with fresh 100% ethanol.
3. Air dry slides for 2-5 minutes, if desired.
4. Immerse slides in pre-warmed Pretreatment Solution at 80° C. for 10 minutes.
5. Immerse slides in purified water at RT for 3 minute.

Protease Pretreatment
1. Immerse slides in Protease Solution at 37° C. for 10-60 minutes (depending on the condition of samples) and monitor the condition of cells under a light microscope.
2. Immerse slides in purified water at RT for 3 minutes.
3. Air dry slides for 2-5 minutes.

Slide Dehydration
1. Immerse slides in 70% ethanol for 3 minutes.
2. Immerse slides in 90% ethanol for 3 minutes.
3. Immerse slides in 100% ethanol for 3 minutes.
4. Air dry slides.

Probe Preparation
1. Pre-warm the probe at RT for 20-30 minutes.
2. Briefly vortex and spin down the probe.

Co-denaturation & Hybridization
1. Apply 10 μl of the probe on each hybridization area and cover with a 22 mm×22 mm coverslip. Seal coverslip(s) with rubber cement.
2. Co-denature slides with probe at 72° C. for 5 minutes.
3. Place slides in a pre-warmed humidified hybridization chamber and incubate slides at 37° C. overnight (16 hours).

Post-Hybridization Wash
1. Mark each hybridization area on the back of the slides with a diamond-tip pen.
2. Carefully remove rubber cement.
3. Immerse slides in Post-hybridization Wash Solution at RT to loosen the coverslips. Shake gently to remove the coverslips; do not pull the coverslips off.
4. Immerse slides in pre-warmed Post-hybridization Wash Solution at 72° C. for 2 minutes.

Slide Dehydration
1. Immerse slides in 70% ethanol for 2 minutes.
2. Immerse slides in 90% ethanol for 2 minutes.
3. Immerse slides in 100% ethanol for 2 minutes.
4. Air dry slides in the dark.

Visualization
1. Apply DAPI counterstain and cover slides with coverslips.
2. Examine slides under a fluorescence microscope with proper filter sets.

All publications cited herein are incorporated by reference in their entirely for the disclosure for which they are cited.

In this specification, “a” and “an” and similar phrases are to be interpreted as “at least one” and “one or more.” References to “an” embodiment in this disclosure are not necessarily to the same embodiment.

The disclosure of this patent document incorporates material which is subject to copyright protection. The copyright owner has no objection to the facsimile reproduction by anyone of the patent document or the patent disclosure, as it appears in the Patent and Trademark Office patent file or records, for the limited purposes required by law, but otherwise reserves all copyright rights whatsoever.

While various embodiments have been described above, it should be understood that they have been presented by way of example, and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. Thus, the present embodiments should not be limited by any of the above described exemplary embodiments.

Further, the purpose of the Abstract of the Disclosure is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract of the Disclosure is not intended to be limiting as to the scope in any way.

Finally, it is the applicant's intent that only claims that include the express language “means for” or “step for” be interpreted under 35 U.S.C. 112, paragraph 6. Claims that do not expressly include the phrase “means for” or “step for” are not to be interpreted under 35 U.S.C. 112, paragraph 6.

What is claimed is:

1. A method of detecting prostate cancer in a human male, comprising hybridizing an isolated nucleic acid probe to a chromosome present in the prostate tissue of a human male, where the probe detects a deletion in the PTEN gene, and
where the probe comprises at least two different nucleic acid molecules comprising DNA from the PTEN gene, where the amounts of each nucleic acid molecule present in the probe are in different proportions from each other and in different proportions in which they are present in the gene as it occurs in nature, and where the DNA in the probe is detectably labeled, and where the nucleic acid molecules are selected from the list in FIG. 4.
